(19) United States
(12) Patent Application Publication
(10) Pub. No.: US 2018/0153924 A1
(43) Pub. Date: Jun. 7, 2018

(54) COMPOSITIONS AND METHODS FOR AQUACULTURE

(71) Applicant: WISCONSIN ALUMNI RESEARCH FOUNDATION, Madison, WI (US)

(72) Inventor: TERENCE P. BARRY, MIDDLETON, WI (US)

(21) Appl. No.: 15/834,442

(22) Filed: Dec. 7, 2017

Related U.S. Application Data
(60) Provisional application No. 62/431,179, filed on Dec. 7, 2016.
(60) Provisional application No. 62/573,365, filed on Oct. 17, 2017.

(51) Int. Cl. A61K 31/737; A23K 50/80; A23K 20/10

(52) U.S. Cl. CPC A61K 31/737; A23K 20/10; A23K 50/80

(57) ABSTRACT

Methods and compositions for increasing the growth rate of a prey fish are described. A method includes contacting the prey fish with a water-soluble growth promoting factor in an amount effective to increase the growth rate of the prey fish. The water-soluble growth promoting factor can be released through fish skin from the urine and/or feces of a predator fish that has eaten a prey fish. An aquaculture growth supplement includes a carrier and a water-soluble growth promoting factor. Also included are methods of making aquaculture growth supplements.

### CROSS-REFERENCE TO RELATED APPLICATIONS

This application claims priority to U.S. Provisional Application 62/431,179 filed on Dec. 7, 2016, and U.S. Provisional Application 62/573,365, filed on Oct. 17, 2017, which are incorporated herein by reference in their entirety.

### FIELD OF THE DISCLOSURE

The present disclosure is related to compositions and methods useful in aquaculture, particularly those that improve the growth rates of fish.

### BACKGROUND

Aquaculture is a form of agriculture that involves the propagation, cultivation and marketing of aquatic animals and plants in a controlled environment. The aquaculture industry is currently the fastest growing food production sector in the world. Aquaculture is one of a range of technologies needed to meet increasing global demand for seafood, support commercial and recreational fisheries, and restore species and marine habitat.

As in all animal agriculture, maximizing the growth rates of aquacultured fish is essential to creating a profitable production enterprise. There are numerous methods to increase fish growth, including genetic selection, the use of superior feed formulations, flavor enhancers, and probiotics, as well as biotechnological approaches, such as the use of transgenic fish, hybrid fish, and monosex fish populations. Most of these approaches, however, are species specific or have other drawbacks that limit their widespread utilization. As such, improved compositions and methods for increasing fish production are of interest, particularly those that can increase the growth rate of fish.

### BRIEF SUMMARY

In an aspect, a method of increasing the growth rate of a first prey fish comprises contacting the first prey fish with a water-soluble growth promoting factor in an amount effective to increase the growth rate of the first prey fish, wherein the water-soluble growth promoting factor is released through fish skin, or the water-soluble growth promoting factor is from the urine and/or feces of a predator fish that has eaten a second prey fish.

In another aspect, an aquaculture growth supplement comprises a carrier and a water-soluble growth promoting factor that is released through fish skin, for example in response to predation or stress.

In another aspect, an aquaculture growth supplement comprises a carrier and a water-soluble growth promoting factor that is from the urine and/or feces of a predator fish that has eaten a prey fish.

In yet another aspect, a method of making an aquaculture growth supplement comprises extracting a water-soluble growth promoting factor from fish skin and adding a carrier to the water-soluble growth promoting factor to provide the aquaculture growth supplement.

In yet another aspect, a method of making an aquaculture growth supplement comprises isolating a water-soluble growth promoting factor that is from the urine and/or feces of a predator fish that has eaten a prey fish, and adding a carrier to the water-soluble growth promoting factor to provide the aquaculture growth supplement.

### BRIEF DESCRIPTION OF THE DRAWINGS

FIG. 1 shows the mean±s.e. mass (M) of Perca flavescens from three treatment groups (n=30 P. flavescens sampled from each raceway at each time point; raceways initially contained 214 P. flavescens): control (▴), a single Sander vitreus in the raceway with the P. flavescens (▪), and a single S. vitreus held in a 110 l tank located above the raceway (●). In this latter treatment, the S. vitreus was fed P. flavescens daily to satiation, and the aquarium discharged into the raceway below. At 3 weeks, the S. vitreus in the raceway was replaced with a larger individual because no P. flavescens had been eaten during the first 3 week interval. There was only one raceway per treatment thus data are individual P. flavescens from the same raceway. As there was no raceway replication, no statistics were performed.

FIG. 2 shows the tank design for example 2 with four treatment groups, each replicated three times: (a) treatment 1, control; (b) treatment 2, hybrid Sander vitreus fed Perca flavescens; (c) treatment 3, hybrid S. vitreus fed dry fish feed; (d) treatment 4, hybrid S. vitreus fed Pimephales promelas.

FIG. 3 shows the mean±s.e. mass (M) of Perca flavescens from four treatment groups (n=3 tanks with c. 250 fish per tank): treatment 1, control (□); treatment 2, hybrid Sander vitreus fed P. flavescens (●); treatment 3, hybrid S. vitreus fed formulated diet (▪); treatment 4, hybrid S. vitreus fed Pimephales promelas (◯). * P<0.05 v. control; ** P<0.01 v. control within sampling time.

The above-described and other features will be appreciated and understood by those skilled in the art from the following detailed description, drawings, and appended claims.

### DETAILED DESCRIPTION

The inventors of the present application unexpectedly discovered that when a walleye was added to a tank containing yellow perch (Perca flavescens), the surviving yellow perch grew twice as fast as yellow perch in an adjacent tank that did not contain walleye predators. A follow-up experiment using an experimental design as shown in FIG. 2 indicated that a water-soluble “predatory factor” (PF) associated with walleye predation on yellow perch was responsible for stimulating the growth of the surviving yellow perch. It is well known that “alarm substances” released from the skin of prey fish can alert conspecifics to danger. There is also limited evidence that such alarm factors can induce changes in body shape in prey fish so that they are less susceptible to predation. There are no reports, however, of odors that can increase the overall growth rate of cultured fish.

In an embodiment, and without being held to a particular theory, it is believed that a water-soluble growth promoting factor is released from the skin of the prey fish upon walleye predation.

In another embodiment, without being held to theory, the water-soluble growth factor is found in the urine and/or feces of the predator fish that has eaten a prey fish. Substances present in the urine and/or feces of the predator fish would be released into the water and available to the olfactory system of fish.

The water-soluble growth promoting factor can increase the growth of perch by as much as 40-110%. Contacting prey fish with this water-soluble growth promoting factor can increase the growth rate of the prey fish. The improvement of growth rate can be observed over one week, 2 weeks, one month, 2 months or longer as, for the most part, fish can grow indefinitely.

In an aspect, a method of increasing the growth rate of a prey fish comprises contacting the prey fish with a water-soluble growth promoting factor in an amount effective to increase the growth rate of the prey fish, wherein the water-soluble growth promoting factor is released through fish skin. In an aspect, the water-soluble growth promoting factor is released through fish skin in response to predation or stress. In another aspect, a method of increasing the growth rate of a first prey fish comprises contacting the first prey fish with a water-soluble growth promoting factor in an amount effective to increase the growth rate of the first prey fish, wherein the water-soluble growth promoting factor is found in the urine and/or feces of a predator fish that has eaten a second prey fish.

In an aspect, the prey fish, e.g., the first prey fish, is contacted with the water-soluble growth promoting factor once or twice daily. Without being held to a particular theory, it is believed that frequent administration of the water-soluble growth promoting factor may cause adaptation among the fish.

As used herein, the term fish refers to vertebrate finfish, including high valued aquaculture species such as carp, tilapia, hybrid striped bass, salmon, trout, catfish, yellow perch, walleye, hybrid walleye, bluegills, bass, surubim, milkfish, mullets, cod, cobia, sea bass, snappers, tuna, sea bream, and sole, for example, and the like. The term prey fish refers to fish that are preyed on by larger predator fish for food.

Exemplary prey fish/predator pairs include yellow perch/walleye, yellow perch/northern pike, walleye/northern pike, Atlantic salmon/walleye, Atlantic salmon/pike, rainbow trout/walleye, rainbow trout/pike, tilapia/walleye, tilapia/pike, and the like.

The water-soluble growth-promoting factor increases the growth rate of growing fish. The term growing fish means fish that are increasing in length and weight in time. Exemplary growing fish are larval, juvenile and adult fish. Juvenile fish are fish that can eat on their own, but have not yet developed into reproductively mature adults. New methods to improve fish growth and survival are needed, particularly for aquaculture that can improve growth and that are water-soluble. Growth can be measured as the length of the fish, such as the average length of fish in a population, or by weight.

In a specific aspect, the fish is in aquaculture. As used herein, aquaculture means the active cultivation of aquatic organisms under controlled conditions. Aquaculture systems use water as the medium for cultivation. An aquaculture system must provide clean and oxygenated water to support the cultivated organisms as well as a means to remove deoxygenated water and wastes. As used herein, aquaculture includes both marine and freshwater aquaculture. Typical aquaculture systems include holding tanks and means for filtering, dissolved gas control, and temperature control.

In an embodiment, the water-soluble growth promoting factor is in the form of a skin extract from a fish. In an aspect, the fish subjected to predation or stress.

In an aspect, a method to prepare a skin extract is to make multiple short, shallow cuts that break the dermis of the prey fish without drawing blood. The fish is then placed into a container of water and the water mixed and agitated so that water-soluble growth-promoting factors in the skin are released into the water. The extract is then frozen to preserve the active chemical(s). Alternatively, the skins of some species of fish are removed by hand or machine during processing and these skins can be collected and processed to obtain the active chemical(s).

In an embodiment the skin extract is from the same species as the prey fish. In another embodiment, the skin extract is from a different species than the prey fish (e.g., a fathead minnow).

In an embodiment, the water-soluble growth promoting factor is an alarm pheromone such as chondroitin-sulfate or hypoxanthine-3-N-oxide. In an aspect, the chondroitin-sulfate is isolated from a skin extract from fish.

Without being held to a particular theory, it is believed that water-soluble, growth-promoting fragments of chondroitin-sulfate with different sulfation patterns are released from the skin of the prey when the prey is bitten by a predator and these chemicals act to increase the growth in the prey that smell the chemicals. Chondroitin-sulfate is a long-chain sulfated glycosaminoglycan polymer consisting of repeating units of alternating sugars (N-acetylgalactosamine and glucuronic acid) with a variety of sulfation patterns. Without being held to a particular theory, it is believed that this polymer can be cleaved by enzymes in the skin into smaller fragments when the skin is disturbed by predation. Chondroitin-sulfate has the following formula

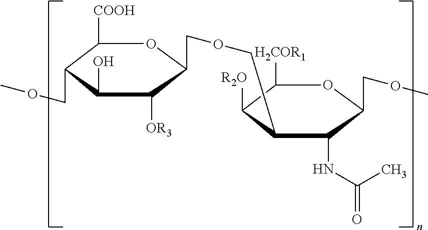

wherein n is number of repeating disaccharide units (a chain can have over 100 individual sugars in some polymers, that is, n can be greater than 50) and R₁, R₂ and R₃ in each disaccharide unit of the molecule are each independently H or SO₃H. In certain embodiments, the chondroitin-sulfate comprises 0-100% of one or more of chondroitin-4-sulfate, chondroitin-6-sulfate, chondroitin-2,6-sulfate and/or chondroitin-4,6-sulfate. Exemplary effective amounts of the water-soluble growth promoting factor to increase the growth rate of the prey fish are 1 to 1000 mg/L crude skin extract.

In another embodiment, the water-soluble growth promoting factor is a stress hormone such as cortisol, a metabolite of cortisol, bile acids, or a combination thereof. A stress hormone such as cortisol or its metabolites or bile acids could be present, for example, in feces or urine as a conjugated metabolite.

In an embodiment, contacting the prey fish with a water-soluble growth promoting factor in an amount effective to increase the growth rate of the prey fish comprises growing the prey fish in a tank comprising a predator for the prey fish, wherein the predator is of sufficient size to feed on the prey fish. For example, growing yellow perch in the presence of walleye of sufficient size to feed on the yellow perch increases the growth rate of the surviving yellow perch.

In another embodiment, contacting the prey fish with a water-soluble growth promoting factor in an amount effective to increase the growth rate of the prey fish comprises contacting the prey fish with water from a tank in which prey fish were exposed to a predator. Without being held to a particular theory, it is believed that the water-soluble growth factor is released from the skin of the prey fish into the water or is found in the urine and/or feces of the predator fish that has eaten a prey fish, which would also be released into the water.

In an embodiment, an aquaculture growth supplement comprises a carrier and a water-soluble growth promoting factor that is released through fish skin, or is found in the urine and/or feces of the predator fish that has eaten a prey fish.

Exemplary carriers include liquid and solid pharmaceutically acceptable carriers such as water, water-soluble solvents, lactose, sugar, maize-starch, calcium phosphate, sorbitol or glycine.

In addition to the water-soluble growth promoting factor, the aquaculture growth supplement can include salts, probiotics, and the like.

In an aspect, a method of making an aquaculture growth supplement comprises extracting a water-soluble growth promoting factor from fish skin, and adding a carrier to the water-soluble growth promoting factor to provide the aquaculture growth supplement. In another aspect, method of making an aquaculture growth supplement comprises isolating a water-soluble growth promoting factor from urine and/or feces of a predator fish that has eaten a prey fish, and adding a carrier to the water-soluble growth promoting factor to provide the aquaculture growth supplement.

The invention is further illustrated by the following examples.

### EXAMPLES

### Methods

### General Procedures

Facilities. Tank studies can be conducted in facilities of the UW Aquaculture Research Laboratory which uses tempered, dechlorinated, flow-through City of Madison, Wis. water. Water temperature will optimal for the growth of the fish species (e.g., 21-24° C. for yellow perch and 15° C. for salmonids). Flow rates will be adjusted to ensure at least 15 turnovers per day. All tanks will be aerated. The photoperiod will be adjusted to ensure optimal growth (e.g., 16L:8D and light intensity will be kept under 100 lumens for yellow perch). The pond studies may be conducted at Coolwater Farms (CWF), LLC, Cambridge, Wis.

Fish. Yellow perch Perca flavescens were produced using intensive larval rearing methods with eggs obtained from Coolwater Farms, LLC (Cambridge, Wis.). The P. flavescens were reared in 750 l tanks supplied with carbon-filtered city water. All growth experiments were conducted at 21° C. at a photoperiod of 16:8 h light:dark. Water variables were: flow rate, 1.5 exchanges h−1; dissolved oxygen, 8.2-8.8 mg l−1; pH, 7.2; hardness, 300 mg/l. The P. flavescens were fed two or three times daily to apparent satiation using Skretting Gemma diets (www.skrettingusa.com). The predators were wild purebred walleye S. vitreus captured by angling from Lake Mendota (Madison, Wis.) in example 1 and tank-reared hybrid S. vitreus [male sauger Sander canadensis (Griffith & Smith1834)×female S. vitreus] produced intensively in the laboratory and trained to eat formulated diets in example 2. Fathead minnows Pimephales promelas Rafinesque1820 were obtained from a colony maintained at the Wisconsin State Laboratory of Hygiene (Madison, Wis.).

### Example 1

### Effects of Walleye Predation on the Growth of Yellow Perch

Experiment 1 was conducted to determine if a water-soluble factor associated with S. vitreus predation, such as an alarm cue, stimulated the growth of P. flavescens. Perca flavescens (n=214 per tank, mean±S.E. mass, M=5.2±0.2 g, 1 year old) were reared in each of three 600 l raceways. There were three treatments: control (no S. vitreus); one S. vitreus (29.4 g, but replaced with a 135.7 g S. vitreus after week 3) in the same tank with the P. flavescens and third, one S. vitreus (32.4 g) in a 110 l round tank situated above the raceway where the water discharged into the raceway below and the S. vitreus was fed one P. flavescens daily. All the P. flavescens in each tank were counted and their total masses measured at times 0, 3 and 6weeks. Individual M and total length (LT) were also measured in random subsamples of c. 10% of the P. flavescens at each sampling time to determine condition (100 MLT⁻³).

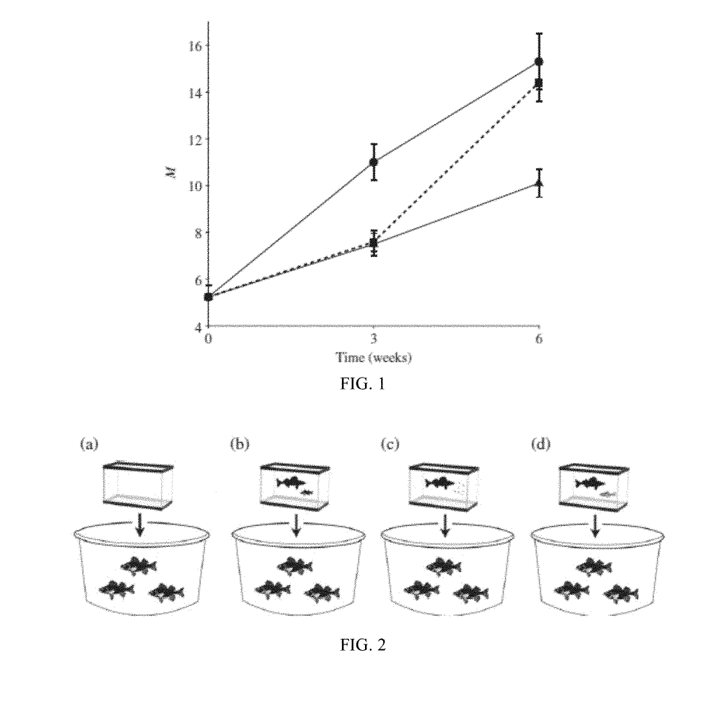

No statistical analysis was performed as there was only one raceway per treatment. Results are reported as means±S.E. mass of the individually weighed P. flavescens. On week three, the S. vitreus in treatment 3 (S. vitreus in the same tank with P. flavescens) had not yet eaten (i.e. there was 100% survival of P. flavescens) and the small S. vitreus was replaced with a larger specimen. Following this change, the growth rate of P. flavescens in treatment 3 markedly increased (FIG. 1). By week 6, the P. flavescens exposed to S. vitreus predation in treatments 2 and 3 were 51 and 42% larger than the P. flavescens in the control tank, respectively. Survival at week 6 in treatments 1, 2 and 3 was 96.3, 93.9 and 78.5%, respectively, indicating that P. flavescens in treatment 3 had been eaten by the larger S. vitreus. Perca flavescens condition averaged 1.94±0.03 across all treatment groups at week 6.

### Example 2

### The Growth-Promoting Effect of S. vitreus Predation on P. flavescens Required Live Prey

Juvenile P. flavescens (n=236-259 per tank, initial mean±S.E. M=0.85±0.03 g, equal initial biomass in each tank) were randomly distributed into 12, 110 l flow-through round fibreglass tanks. A 30 l fibreglass aquarium was situated above each of these tanks and plumbed such that the water flowed through the aquaria and discharged into the tanks below. A 150 μm mesh net was used to collect particulate matter (faeces, scales etc.) in the discharge. There were four treatments each replicated three times (FIG. 2): (1) control (no predator); (2) two hybrid S. vitreus in the upper tank fed P. flavescens; (3) two hybrid S. vitreus in the upper tank fed dry formulated diet; (4) two hybrid S. vitreus in the upper tank fed P. promelas. The hybrid S. vitreus ranged in size from 10.2-14.3 g and were fed to satiation daily (up to five P. flavescens or P. promelas were eaten per day). Perca flavescens growth was measured every 2 weeks by individually measuring the LT and M of c. 30% of the population in each tank. The experiment ran for 98 days at which time all the P. flavescens were counted to determine survival and sexed according to the method of Shepherd et al. (2013) to determine if there was a change in the sex ratio favouring fast-growing females. The specific growth rates (GSR=100(lnMt−lnM0)/−1, where M0 is initial mass, Mt is final mass and t is time in days) and condition were calculated. The mass data were analyzed by two-way ANOVA using Stata/SE 15.0 for Mac (StataCorp; www.stata.com). The model included time and treatment as the main effects and the time treatment interaction. Treatment means for the mass, GSR and condition data were compared at each sampling time by one-way ANOVA followed by Fisher's least significant difference test (LSD). Results are reported as means±S.E. of the replicate tanks.

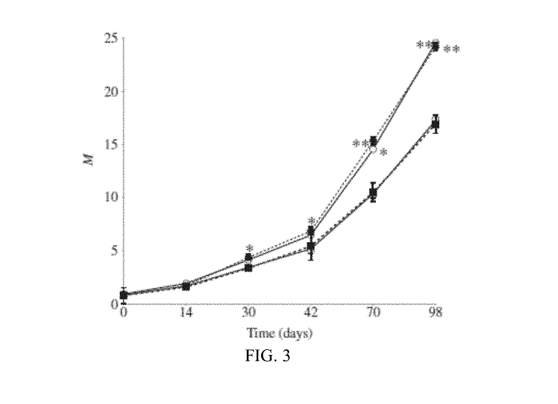

There was a significant interaction between time and treatment on the mass of P. flavescens (two-way ANOVA, F15,48=8.00, P<0.001). At the end of the experiment on day 98, the P. flavescens in treatments 2 (predator fed P. flavescens) and 4 (predator fed P. promelas) were 42 and 40% heavier than the control P. flavescens, respectively (FIG. 3). These mass differences on day 98 were statistically significant (one-way ANOVA, F3,8=15.98, P<0.001; LSD test, d.f.=3, P<0.01). Significant treatment effects on M were also detected on days 30, 42 and 70, FIG. 3). On day 98, the GSR of the P. flavescens in treatments 2 (GSR=3.6% day−1) and 4 (GSR=3.4% day−1) were significantly higher than those in treatments 3 (predator fed dry diet) and 1 (GSR for both=3.1% day−1) (one-way ANOVA, F3,8=17.70, P<0.001; LSD tests, d.f.=3, P<0.01). There was no difference in mass gain between the controls and individuals in treatment 3 (predator fed formulated diet) at any time point (LSD test, d.f.=3, P>0.05). There were no differences in P. flavescens condition or survival among treatments at any time point (LSD tests, d.f.=3, P>0.05). Sex ratios were c. 50:50 in all tanks on day 98. Significant changes in growth first appeared on day 30 when P. flavescens were c. 3.5 g in mass (FIG. 3).

The results suggest that a water-soluble factor associated with S. vitreus predation on P. flavescens and P. promelas can markedly increase the growth rate of P. flavescens. The effect was not due to changes in sex ratio or declining rearing density (example 2) which were possible explanations for the growth of P. flavescens reared in the same tanks as the predator. The data also indicate that the sight or smell of the predator itself is not sufficient to stimulate increased growth in P. flavescens. This conclusion is supported by the observations that the P. flavescens in experiment 1 treatment 3 (S. vitreus in the same tank with P. flavescens), did not show enhanced growth over the first 3 weeks when there was no predation and also that P. flavescens in experiment 2 exposed to odours associated with predators fed formulated diet (treatment 3) did not grow faster than controls. The data suggest that there may be an ontological component to the growth-promoting effect of predation as growth differences were only observed in P. flavescens that were c. 3.5 g in mass.

It is postulated that the putative growth-promoting substance, or substances, acts via an olfactory-endocrine axis. That is, the odour stimulates olfactory neurons that ultimately innervate brain centres regulating the production and release of pituitary growth hormone. A similar olfactory-endocrine axis controls the release of pituitary gonadotropin in response to the sex pheromone 17α,20β-dihydroxy-4-pregnen-3-one in goldfish Carassius auratus.

### Example 3

### Effect of Walleye Predation on Growth of Perch in Ponds

These experiments will be conducted at CWF during their normal fingerling feed-training period. Fish at approximately 0.5 g are harvested from large production ponds and trained to accept formulated diets, first in two large tank for two weeks, and then in multiple small “microponds” for another two weeks. The feed-trained fish are then harvested, size-graded and counted and stocked in production ponds for growout.

The predation experiment will be conduct during the entire one month training period. About 40,000 yellow perch (approximately 0.5 g) are placed into each of the large tanks. One tank will be used as a control (no predator), and the other tank will be stocked with both yellow perch and 400 small hybrid walleyes large enough to prey on the yellow perch (approximately 1 g). After two weeks, the fish will be transferred from the tanks into the ponds. Each pond will receive approximately 20,000 fish. Two of the ponds will be stocked with fish from the control tanks and two ponds with fish from the experimental tanks. The two experimental ponds will each contain approximately 200 small hybrid walleyes (the same fish originally stocked in the tanks). At the end of the 2-week period in the ponds, the fish will be harvested and total fish weight and size distribution will be recorded. It is expected that the yellow perch in the ponds containing the predators will grow more rapidly. Additional experiment will be performed with the predators confined in floating netpens in the tanks and microponds. This experiment will help determine if odors associated with predation can influence the growth of fish.

### Example 4

### Effects of Predation on the Growth of Hybrid Walleyes in Tanks

Eighteen 20-L aquaria will be used (plus six more “above” tanks, N=6). Each tank will be stocked with 20 hybrid walleyes (density of approximately 1 g/L). A single northern pike will be added to the appropriate tanks. Preliminary experiments will be conducted to determine the optimal predator size. The goal is to use fish large enough to eat the prey, but small enough that they do not consume all of the prey before the prey have had a chance to grow. It is expected that the hybrid walleyes in the tanks containing the predators will grow more rapidly.

### Example 5

### Effects of Predation on the Growth of Atlantic Salmon in Tanks

Eighteen 20-L aquaria will be used (plus six more “above” tanks, N=6). Each tank will be stocked with 20 Atlantic salmon (density of ˜1 g/L). A single northern pike or walleye will be added to the appropriate tanks. Preliminary experiments will be conducted to determine the optimal predator size. The goal is to use fish large enough to eat the prey, but small enough that they do not consume all of the prey before the prey have had a chance to grow. It is expected that the Atlantic salmon in the tanks containing the predators will grow more rapidly.

### Example 6

### Identification of Source and Chemical Identification of the Water-Soluble Growth Promoting Factor(s)

Without being held to a particular theory, it is hypothesized that “alarm substances” released by the prey upon predation are the chemicals mediating the observed increases in growth rate. Alarm substances can be rapidly screened by observing and quantifying behavioral fright responses in fish subjected to these chemicals. Thus, a key first step in attempting to identify the active chemicals is to develop a robust and reliable behavioral bioassay that can be used to quickly screen active chemicals. Behavioral alarm responses include things like darting, hiding, and schooling. The activities can be video recorded, quantified and analyzed statistically.

The alarm pheromone is zebrafish has recently been identified as chondroitin-sulfate (CS), and it is possible that this or a similar chemical is the water-soluble growth factor in yellow perch. CS is a sulfated glycosaminoglycan composed of a long chain of alternating sugars (N-acetylgalactosamine and glucuronic acid), is usually found attached to proteins as part of a proteoglycan, and is a constituent of fish skin and mucus. A CS chain can have over 100 individual sugars, each of which can be sulfated in different positions and quantities. When fish skin is broken by predation, enzymes are activated that cleave CS into fragments of various sizes. The heterogeneity of CS chain length and the variability of sulfation patterns can account for the diverse species-specific alarm cues found among fishes. Without being held to a particular theory, it is believed that the water-soluble growth factor in yellow perch is a CS fragment of a specific size and sulfation pattern.

In one behavioral assay, fish are “incubated” for 1-6 minutes in 1-L of water containing a test material and then the side of the beaker is tapped with a small rubber mallet. The control fish (no chemical exposure) show a robust “startle response” over 95% of the time. In contrast, over 85% of the exposed fish do not move in response to the stimulus. The bioassay will be used to test potential candidate chemicals including crude skin extracts of both prey and predator, predator urine, predator feces, and known alarm substances in fish including CS fragments and hypoxanthine-3-N-oxide. Standard biochemical fractionation methods can be used to determine if the compounds of interest in skin extracts, for example, are polar or nonpolar, volatile, and have larger (or small) molecular weight.

For example, if CS is the active chemical, studies using HPLC/MS will be initiated to further investigate CS in yellow perch. We will determine if CS is released into the water when walleye prey on yellow perch, and begin CS fractionation experiments to determine the effects of CS chain length and sulfation pattern on the yellow perch behavioral response.

### Example 7

### Elucidating the Mechanism of Action of the Water-Soluble Growth Factor

Without being held to a particular theory, it is believed that the water-soluble growth factor acts by stimulating the release of growth hormone from the pituitary which in turn stimulates the production and release of IGF-1 from the liver. IGF-1 is the primary growth-promoting hormone in fish acting on numerous targets to increase bone elongation and muscle accretion. Changes in IGF-1 levels are closely correlated with changes in fish growth and in some cases can be used as a surrogate for growth.

A group of 48 small yellow perch (approximately 5 g size) will be exposed to walleye predation and/or skin extracts and six fish will be bled at each of the following time points following exposure: 0, 0.5, 1, 3, 6, 12, 24 and 48 hrs post-exposure to determine when peak IGF-1 levels occur in response to walleye predation or skin extract. We will also conduct experiments to determine if the water-soluble growth factor can increase the expression of pituitary GH or inhibit the expression of somatostatin using PCR.

In some growth experiments, we will measure blood levels of IGF-1 to determine if growth and IGF-1 levels are correlated. It may be possible to use short-term changes in IGF-1 levels in lieu of lengthy and expensive fish growth experiments to determine the effects of the water-soluble growth factor on growth.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of increasing the growth rate of a first prey fish, the method comprising
contacting the first prey fish with a water-soluble growth promoting factor in an amount effective to increase the growth rate of the first prey fish, wherein the water-soluble growth promoting factor is released through fish skin, or the water-soluble growth promoting factor is from the urine and/or feces of a predator fish that has eaten a second prey fish.

2. The method of claim 1, wherein the contacting is done once or twice daily.

3. The method of claim 1, wherein the water-soluble growth promoting factor is released through fish skin in response to predation or stress.

4. The method of claim 1, wherein the prey fish is in aquaculture.

5. The method of claim 1, wherein the prey fish is carp, tilapia, hybrid striped bass, salmon, trout, catfish, yellow perch, walleye, hybrid walleye, bluegill, bass, surubim, milkfish, mullet, cod, cobia, sea bass, snappers, tuna, sea bream, or sole.

6. The method of claim 1, wherein the water-soluble growth promoting factor is in the form of a skin extract from fish.

7. The method of claim 6, wherein the skin extract is from the same or different species as the prey fish.

8. The method of claim 1, wherein the water-soluble growth promoting factor is an alarm pheromone.

9. The method of claim 8, wherein the alarm pheromone is chondroitin-sulfate or hypoxanthine-3-N-oxide.

10. The method of claim 9, wherein the chondroitin-sulfate is specific for fish skin.

11. The method of claim 1, wherein contacting the prey fish with a water-soluble growth promoting factor in an amount effective to increase the growth rate of the prey fish comprises growing the prey fish in a tank comprising a predator for the prey fish, wherein the predator is of sufficient size to feed on the prey fish.

12. The method of claim 1, wherein contacting the prey fish with a water-soluble growth promoting factor in an amount effective to increase the growth rate of the prey fish comprises contacting the prey fish with water from a tank in which prey fish were exposed to a predator.

13. The method of claim 12, wherein the prey fish is yellow perch, and the predator is walleye, or the prey fish is walleye and the predator is northern pike.

14. The method of claim 13, wherein the prey fish is yellow perch, and the predator is walleye, or the prey fish is walleye and the predator is northern pike.

15. The method of claim 1, wherein the water-soluble growth promoting factor is from the urine and/or feces of a predator fish that has eaten a second prey fish.

16. The method of claim 1, wherein the water-soluble growth promoting factor is a stress hormone.

17. An aquaculture growth supplement comprises a carrier and a water-soluble growth promoting factor that is released through fish skin.

18. The aquaculture growth supplement of claim 17, wherein the water-soluble growth promoting factor is an alarm pheromone.

19. The aquaculture growth supplement of claim 18, wherein the alarm pheromone is a chondroitin-sulfate or hypoxanthine-3-N-oxide.

20. The aquaculture growth supplement of claim 20, wherein the chondroitin-sulfate is specific for fish skin.

21. An aquaculture growth supplement comprising a carrier and a water-soluble growth promoting factor that is from the urine and/or feces of a predator fish that has eaten a prey fish.
